(19) United States
(12) Patent Application Publication
(10) Pub. No.: US 2024/0350695 A1
(43) Pub. Date: Oct. 24, 2024

(54) MULTIFUNCTIONAL AROMA-DIFFUSOR

(71) Applicant: MMAS LLC, CARY, NC (US)

(72) Inventor: XIAOYUAN MA, Cary, NC (US)

(21) Appl. No.: 18/303,756

(22) Filed: Apr. 20, 2023

(51) Int. Cl. A61L 9/14; A61L 9/12

(52) U.S. Cl. CPC A61L 9/14; A61L 9/122; A61L 2209/12

(57) ABSTRACT

A multifunctional aroma-diffuser, comprising a housing, an lighting auxiliary component, a lamp, an aromatherapy component and a control panel; a storage cavity and a liquid storage cavity are formed in the housing, and a light transmission opening and a mist discharge port are also provided on the housing; the lighting auxiliary components comprise a projection lens, a refracting lens and a motor; the projection lens is arranged at the light transmission opening, the refracting lens is arranged in the storage cavity and corresponds to the projection lens; a motor is fixed in the storage cavity and is configured to drive the refracting lens to rotate; the lamp is arranged in the storage cavity and its light passes through the refracting lens and the projection lens in sequence; the aromatherapy component is arranged in the housing and configured to atomize the essential oil mixture in the liquid storage cavity.

### TECHNICAL FIELD

The present application belongs to the technical field of aroma-diffuser, and in particular relates to a multifunctional aroma-diffuser.

### BACKGROUND

The aroma-diffuser is an aroma diffuser that uses an atomization device to decompose the dissolved plant essential oil mixture into a cold mist and emit it into the surrounding air. It has the functions of purifying the air and improving the environment. The traditional aromatherapy machine has a single function, which is difficult to meet the needs of modern people.

In order to improve the availability of aromatherapy machines, aromatherapy machines with lamps came into being. In addition to the basic function of diffuser, this type of aromatherapy machine can also emit light to enhance the atmosphere. However, the applicant found in actual use that this type of aroma diffuser has the following defects:
First, the light emitted by the lamp is in a static state, not dynamic, and the lighting effect is poor;
Second, because the lamp is installed inside the aroma-diffuser, it is blocked by the housing of the aroma-diffuser, so it can only irradiate through light in a single direction, and the light irradiation range is small.

The above-mentioned defects make the light emitted by the lamps unable to be fully utilized, which leads to a poor ability to render the atmosphere. Therefore, it is necessary to design a multifunctional aroma-diffuser that can improve the ability to render the atmosphere.

### SUMMARY

### (1) Technical Problems to be Solved

The present application provides a multifunctional aroma-diffuser, which not only has the basic function of diffusing fragrance, but also can enhance the lighting effect, expand the irradiation range, and improve the ability to render the atmosphere.

### (2) Technical Solution

In order to achieve the above object, the present application provides the following technical solutions:

A multifunctional aroma-diffuser comprises a housing, an lighting auxiliary component, a lamp, an aromatherapy component and a control panel; a storage cavity and a liquid storage cavity are formed in the housing, and a light transmission opening and a mist discharge port corresponding to the liquid storage cavity are also provided on the housing; the lighting auxiliary component comprises a projection lens, a refracting lens and a motor; the projection lens is arranged in the light transmission opening, the refracting lens is horizontally arranged in the storage cavity and corresponds to the projection lens; the motor is fixed in the storage cavity and is drivingly connected to the refracting lens, so as to drive the refracting lens to rotate; the lamp is arranged in the storage cavity, and light emitted by the lamp passes through the refracting lens and the projection lens in sequence; the aromatherapy component is arranged in the housing and configured to atomize the essential oil mixture in the liquid storage cavity; the control panel is arranged in the storage cavity and is electrically connected with the motor, the lamp and the aromatherapy component.

Preferably, the projection lens and the refracting lens are both made of transparent plastic or glass material; the refracting lens is provided with a plurality of integrally formed refracting blocks; the projection lens is a hemispherical sheet, and a plurality of refracting grooves are uniformly arranged on the projection lens.

Preferably, the housing comprises a base and a cover, the storage cavity and the liquid storage cavity are arranged on the base, and the base is provided with a through port connected to the storage cavity and a liquid inlet connected to the liquid storage cavity; the light transmission opening and the mist discharge port is arranged on the cover, and the cover is detachably connected to the base to cover the port and the liquid inlet port, so that the through port and liquid inlet port respectively correspond to the light transmission opening and the mist discharge port; the motor is fixed in the storage cavity and placed at the through port, and the refracting lens is arranged on an output shaft of the motor and corresponds to the through port, so that the light from the lamp can be smoothly irradiated onto the projection lens arranged at the light transmission opening.

Preferably, the lighting auxiliary component further comprises a fixing frame, and the fixing frame is arranged in the storage cavity and placed at the through port, the motor is arranged on the fixing frame.

Preferably, the lighting auxiliary component further comprises a sealing lens, and the sealing lens is arranged at the through port to block the through port; the light generated by the lamp sequentially passes through the refracting lens and the sealing lens to the projection lens.

Preferably, the lamp is a condenser lens with LED lamps.

Preferably, the aromatherapy component comprises a bracket and an atomizing sheet, and an oil hole is provided on bottom of the liquid storage cavity; the bracket is arranged in the storage cavity and placed at the oil hole, the atomizing sheet is arranged on the bracket and blocks the oil hole; the atomizing sheet is also electrically connected with the control panel to atomize the essential oil mixture entering the oil hole, and the fragrance mist formed after the essential oil mixture is atomized is discharged to the outside through the mist discharge port.

Preferably, the aromatherapy component further comprises a fan, and a cavity cover is arranged on the cover, and the cavity cover is provided with an air nozzle connected to the mist discharge port; when the cover is connected to the base, the cavity cover blocks the liquid inlet port, and the cover and the base enclose to form an air flow cavity; the base is provided with an air guide hole connecting the storage cavity and the air flow cavity, and the cavity cover is provided with an air introduction port connecting the liquid storage cavity and the air flow cavity; the base is also provided with an air inlet slot connecting the outside and the storage cavity; the fan is arranged in the storage cavity and placed beside the air inlet slot, the control panel is electrically connected to the fan; the fan is configured to pump the air from the outside into the liquid storage cavity, the fragrance mist in the liquid storage cavity can flow from the air nozzle and the mist discharge port to the outside.

Preferably, it comprises a sounding component, and the housing is also provided with a sound amplification slot connecting the outside and the storage cavity; the sounding component is arranged in the storage cavity and is placed beside the sound amplification slot; the control panel is electrically connected to the sounding component.

Preferably, a charging socket and a plurality of control buttons are also provided on the housing, and the charging socket is electrically connected with the control panel to supply power to the control panel; the control button is electrically connected to the control panel to control the operation state of the aromatherapy component, the motor, the lamp and the sounding component.

### (3) Beneficial Effect

In a multifunctional aroma-diffuser provided by the present application, the aroma-diffuser component is designed to atomize the essential oil mixture in the liquid storage cavity, so that it has the basic function of expanding aroma, and the lamp is designed to emit light to render the atmosphere; The lighting auxiliary component is used to drive the refracting lens to rotate through the motor, so that the light emitted by the lamp has a dynamic effect and enhance the lighting effect. Then the light is projected from all directions to the outside by the projection lens to expand the lighting range, which is convenient for users to view the dynamic light from all directions, ensures that the light emitted by the lamp can be fully utilized, and greatly improves the ability to render the atmosphere.

### BRIEF DESCRIPTION OF DRAWINGS

The accompanying drawings are used to provide a further understanding of the application, and constitute a part of the description, and are used to explain the application together with the embodiments of the application, and do not constitute a limitation to the application. In the accompanying drawings:

FIG. 1 shows an overall structural schematic diagram of the present application;

FIG. 2 shows a right side view of FIG. 2;

FIG. 3 shows a sectional view A-A of FIG. 2;

FIG. 4 shows a schematic diagram of the present application in a use state;

FIG. 5 shows a partial structural schematic diagram of the present application;

FIG. 6 shows an exploded schematic diagram 1 of the overall structure of the present application;

FIG. 7 shows the second exploded view of the overall structure of the present application;

FIG. 8 shows a schematic structural diagram of the lighting auxiliary component of the present application.

Reference signs: 1 Housing, 101 Storage cavity, 102 Liquid storage cavity, 102a oil hole, 103 Light transmission opening, 104 Mist discharge port, 11 Base, 111 Through port, 112 Liquid inlet port, 11d Air guide hole, 12 Cover, 120 Air flow cavity, 1a Air inlet slot, 1b Sound amplification slot, 2 Lighting auxiliary components, 21 Projection lens, 210 Refracting slot, 22 Refracting lens, 220 Refracting block, 23 Motor, 24 Fixing frame, 25 Sealing lens, 3 Lamp, 4 Aromatherapy components, 41 Bracket, 42 Atomizing sheet, 43 Fan, 5 Control panel, 51 Charging socket, 52 Control button, 6 Cavity cover, 60 Air nozzle, 6d air introduction port, 7 Sounding component.

### DETAILED DESCRIPTION

The technical solutions in the embodiments of the present application will be clearly and completely described below in conjunction with the drawings in the embodiments of the present application. Apparently, the described embodiments are only some of the embodiments of this application, not all of them. Based on the embodiments in this application, all other embodiments obtained by persons of ordinary skill in the art without making creative efforts belong to the scope of protection of this application.

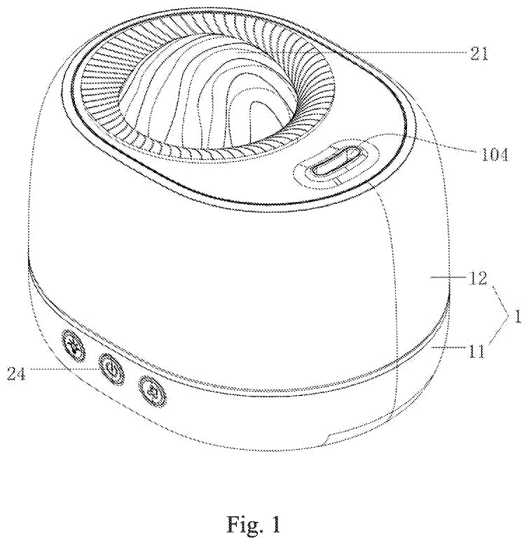

Referring to FIG. 1-4, a multifunctional aroma-diffuser is shown, it comprises a housing 1, an lighting auxiliary component 2, a lamp 3, an aromatherapy component 4 and a control panel 5; a storage cavity 101 and a liquid storage cavity 102 for storing essential oil mixtures are formed in the housing 1, and a light transmission opening 103 and a mist discharge port 104 corresponding to the liquid storage cavity 102 are also provided on the housing 1; the lighting auxiliary component 2 comprises a projection lens 21, a refracting lens 22 and a motor 23; the projection lens 21 is arranged in the light transmission opening 103, the refracting lens 22 is horizontally arranged in the storage cavity 101 and corresponds to the projection lens 21; the motor 23 is fixed in the storage cavity 101 and is drivingly connected to the refracting lens 22, so as to drive the refracting lens 22 to rotate; the lamp 3 is arranged in the storage cavity 101, and light emitted by the lamp 3 passes through the refracting lens 22 and the projection lens 21 in sequence; the aromatherapy component 4 is arranged in the housing 1 and configured to atomize the essential oil mixture in the liquid storage cavity 102; the control panel 5 is arranged in the storage cavity 101 and is electrically connected with the motor 23, the lamp 3 and the aromatherapy component 4.

Specifically, when in use, after ensuring that the essential oil mixture is stored in the liquid storage cavity 102, the aromatherapy component 4 is activated through the control panel 5 to atomize the essential oil mixture in the liquid storage cavity 102. Fragrant mist generated by atomization rises and is discharged to the outside through the mist discharge port 104. Then start the light fixture 3 through the control panel 5 to make it emit light, and then the motor 23 is started by the control panel 5 to drive the refracting lens 22 to rotate continuously, so that the light on the refracting lens 22 is continuously refracted. The refracted light is irradiated onto the projection lens 21, and the projection lens 21 can project the light to various directions.

In summary, this application designs the aromatherapy component 4 to atomize the essential oil mixture in the liquid storage cavity 102, so that it has the basic function of expanding fragrance, and designs the lamp 3 to emit light to render the atmosphere. The lighting auxiliary component 2 uses the design of the motor 23 to drive the refracting lens 22 to rotate, so that the light emitted by the lamp 3 is dynamic and enhances the lighting effect, and then the projection lens 21 projects the light from all directions to the outside world to expand the lighting range. The lighting auxiliary component 2 drives the refracting lens 22 to rotate through the motor 23, so that the light emitted by the lamp 3 is dynamic and enhances the lighting effect, and then the projection lens 21 projects the light to the outside from various directions to expand the lighting range. Therefore, it is convenient for the user to view the dynamic light from various directions, ensuring that the light emitted by the lamp 3 can be fully utilized, and greatly improving the ability to render the atmosphere.

An essential oil mixture is a solution of essential oils mixed with water or other liquids.

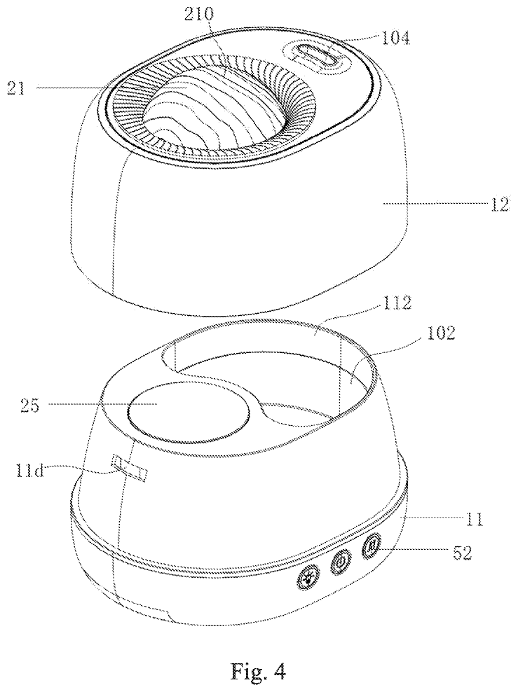

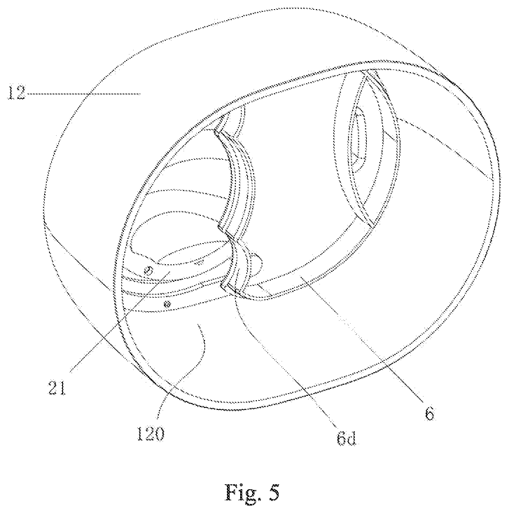

Referring to FIG. 4-8, the projection lens 21 and refracting lens 22 all made of transparent plastics or glass material; the refracting lens 22 is provided with a plurality of integrally formed refracting blocks 220; Moreover, a plurality of refracting grooves 210 are uniformly provided on the spherical surface of the projection lens 21.

Specifically, after the light emitted by the lamp 3 is irradiated on the refracting lens 22, it will correspond to the corresponding part of the refracting blocks 220, and due to the rotation of the plurality of refracting blocks 220 and the refracting lens 22, it is constantly moving. Therefore, each refracting block 220 will intermittently correspond to the light one by one, so that the refraction of the light will continue to change; After the light passes through the refracting lens 22 and irradiates the inner spherical surface of the projection lens 21, it finally passes through the plurality of refracting grooves 210 and is projected to the outside from various directions.

The projection lens 21 and the refracting lens 22 are made of transparent plastic or glass material, which can ensure that both have good light transmission; The refracting block 220 on the refracting lens 22 is designed to control the refraction angle of the light to ensure that the refracted light can be irradiated onto the projection lens 21 smoothly. The structural design of the projection lens 21 is used to evenly distribute the light to the plurality of refracting grooves 210 to diffuse the light.

It should be noted that, regarding the shape design of the refracting block 220 and the refracting groove 210, the manufacturer can design according to the actual needs, which is not limited in this application. In addition, both the projection lens 21 and the refracting lens 22 belong to the prior art, and are widely used in daily life, and the principle of refracting and diffusing light will not be further elaborated in this application.

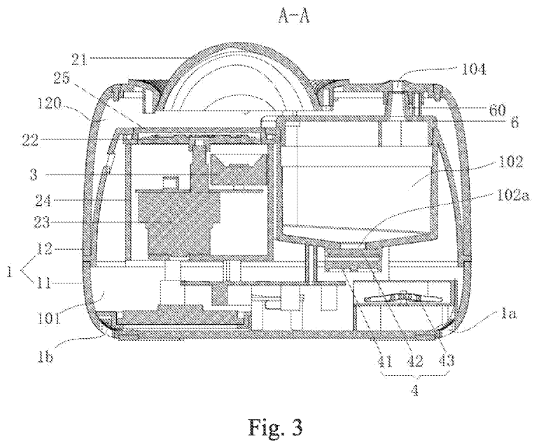

Referring to FIG. 3-8, during actual use, the generated fragrance mist overflows onto the projection lens 21 and condenses on the projection lens 21, affecting the normal use of both; After the essential oil mixture in the liquid storage cavity 102 is used up, it is also inconvenient to add the essential oil mixture; the housing 1 comprises a base 11 and a cover 12, the storage cavity 101 and the liquid storage cavity 102 are arranged on the base 11, and the base 11 is provided with a through port 111 connected to the storage cavity 101 and a liquid inlet 112 connected to the liquid storage cavity 102; the light transmission opening 103 and the mist discharge port 104 is arranged on the cover 12, and the cover 12 is detachably connected to the base 11 to cover the port 111 and the liquid inlet port 112, so that the through port 111 and liquid inlet port 112 respectively correspond to the light transmission opening 103 and the mist discharge port 104; the motor 23 is fixed in the storage cavity 101 and placed at the through port 111, and the refracting lens 22 is arranged on an output shaft of the motor 23 and corresponds to the through port 111, so that the light from the lamp 3 can be smoothly irradiated onto the projection lens 21 arranged at the light transmission opening 103.

Specifically, the housing 1 may be provided with a sleeve groove, and the outer side of the base 11 is fitted through the sleeve groove, so that the two can be disassembled from each other; When in use, the housing 1 is removed from the base 11 to expose the liquid inlet port 112, and the essential oil mixture can be added to the liquid storage cavity 102; At this time, both the upper surface of the refracting lens 22 and the inner surface of the projection lens 21 are exposed, and the user can use a cloth to wipe and maintain.

In summary, the above design not only facilitates the user to add the essential oil mixture into the liquid storage cavity 102, but also facilitates the user to wipe and maintain the refracting lens 22 and the projection lens 21 to prevent the refracting lens 22 and the projection lens 21 from being affected.

Referring to FIG. 3-6, the lighting auxiliary component 2 also comprises fixing frame 24, and fixing frame 24 is arranged in storage cavity 101 and is placed in the through port 111, and motor 23 is arranged on fixing frame 24; The design of fixing frame 24 is used to fix the motor 23 to ensure the stable operation of the motor 23.

Referring to FIG. 3-6, considering that the lower surface of the refracting lens 22 is placed in the storage cavity 101, it is inconvenient to wipe and maintain. In order to solve this problem, in this application, the lighting auxiliary assembly 2 also comprises a sealing lens 25, and the sealing lens 25 is arranged at the through port 111 to block the through port 111; the light generated by the lamp 3 sequentially passes through the refracting lens 22 and the sealing lens 25 to the projection lens 21.

Since the sealing lens 25 blocks the through port 111, the storage cavity 101 can be sealed, thereby isolating the lens 22 and avoiding the essential oil mixture or fragrance mist in the storage cavity from overflowing into the storage cavity 101 and contaminating the refracting lens 22. If the upper surface of the sealing lens 25 is contaminated with essential oil mixture or fragrance mist, first remove the housing 1 from the base 11, and then use a cloth to wipe the upper surface of the sealing lens 25.

Therefore, the design of the sealed lens 25 can reduce the difficulty of cleaning the lens and facilitate the daily maintenance of the user.

It should also be noted that the sealing lens 25 can be made of plastic or transparent plastic or glass material, so that it has good light transmission, so as to ensure that the light emitted by the lamp 3 passes through smoothly.

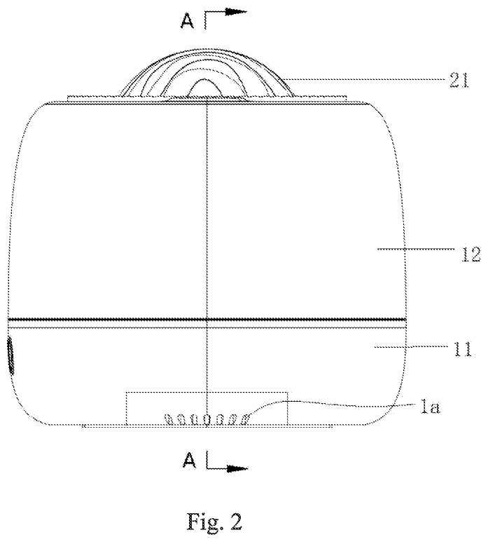

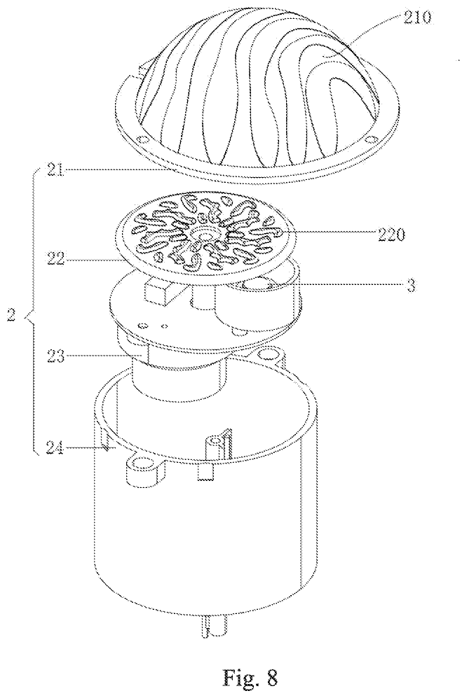

Referring to FIG. 2 and FIG. 8, the lamp 3 is a condenser lens with LED lamps.

LED lamps are used to emit light to create an atmosphere. In this application, LED lamps that can emit light of different colors are preferred; and the condenser lens with LED lamps can concentrate the light to ensure that the light can be accurately irradiated to a specific part of the refracting lens 22 to facilitate the refraction lens 22 to carry out refraction.

Since the condenser lens with LED lights belongs to the prior art, its working principle and specific structure will not be further elaborated in this application.

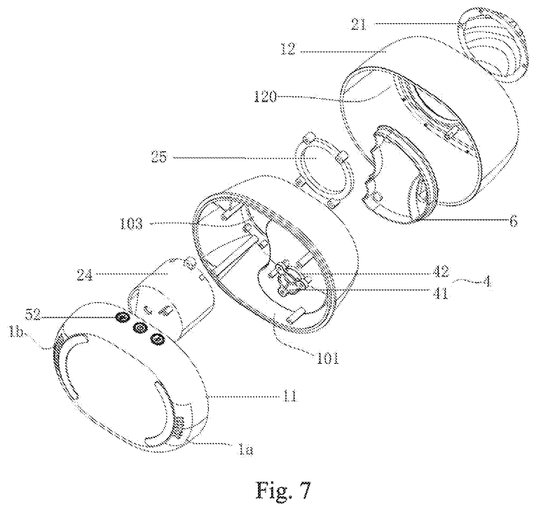

Referring to FIGS. 3 and 7, the aromatherapy component comprises a bracket 41 and an atomizing sheet 42, and an oil hole 102a is provided on bottom of the liquid storage cavity 102; the bracket 41 is arranged in the storage cavity 101 and placed at the oil hole 102a, and the atomizing sheet t 42 is arranged on the bracket 41 and blocks the oil hole 102a; the atomizing sheet 42 is also electrically connected with the control panel 5.

Specifically, the bracket 41 is used to fix the atomizing sheet 42. When in use, the atomizing sheet 42 is activated through the control panel 5 to atomize the essential oil mixture entering the oil hole 102a to generate fragrance mist, and the fragrance mist formed after the essential oil mixture is atomized is discharged to the outside through the mist discharge port 104 and plays an aromatherapy effect.

The atomizing sheet 42 is based on the prior art, and the technology of controlling the operation of the atomizing sheet 42 through the control panel 5 is also very common, which belongs to the technology that should be clear to those skilled in the art, so the specific circuit structure thereof will not be described in detail in this application.

It should be noted that there are various types of aromatherapy components 4. For example, the atomizing sheet 42 can be directly installed at the mist discharge port 104, and use a water-absorbing cotton swab to absorb the essential oil mixture to the atomization sheet 42 to generate a fragrance mist through atomization; Since there are many types of aromatherapy components on the market, which are common technical means, there is no limitation in this application.

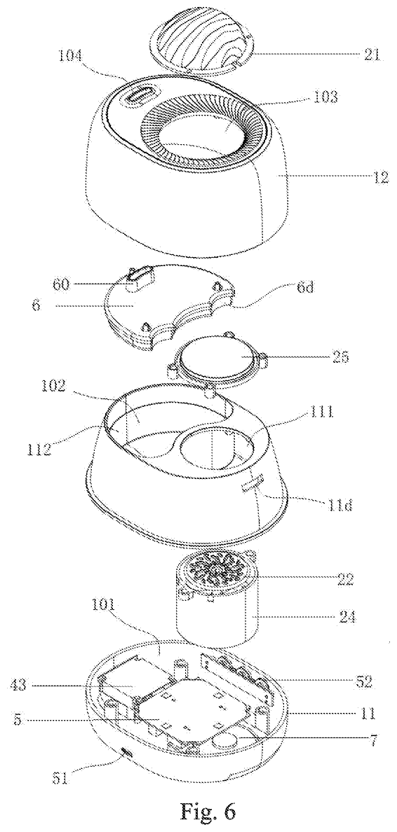

Referring to FIG. 3 and FIG. 6, in this application, the aromatherapy component 4 comprises a fan 43, and a cavity cover 6 is arranged on the cover 12, and the cavity cover 6 is provided with an air nozzle 60 connected to the mist discharge port 104; when the cover 12 is connected to the base 11, the cavity cover 6 blocks the liquid inlet port 112, and the cover 12 and the base 11 enclose to form an air flow cavity 120; the base 11 is provided with an air guide hole 11d connecting the storage cavity 101 and the air flow cavity 120, and the cavity cover 6 is provided with an air introduction port 6d connecting the liquid storage cavity 102 and the air flow cavity 120; the base 11 is also provided with an air inlet slot 1a connecting the outside and the storage cavity 101; the fan 43 is arranged in the storage cavity 101 and placed beside the air inlet slot 1a, the control panel 5 is electrically connected to the fan 43.

Specifically, when in use, start the fan 43 to pump the air from the outside into the storage cavity 101, and then the fan 43 continues to drive the gas in the storage cavity 101 to make it enter through the air guide hole 11d, the air flow cavity 120 and the air introduction port 6d in sequence. In the liquid storage cavity 102, finally, the fragrance mist in the liquid storage cavity 102 is discharged from the air nozzle 60 and the mist discharge port 104 to the outside.

Therefore, the above-mentioned design can make the aroma mist produced by atomization discharge from the mist discharge port 104 to the outside faster, and the user can adjust the speed of the fan 43 through the control panel 5, thereby controlling the overflow speed of the aroma mist, and then hen adjust the concentration of the aroma mist in the use scene, which improves the usability of the multifunctional aroma-diffuser.

Referring to FIG. 3-FIG. 7, it also comprises a sounding component 7, and the housing 1 is also provided with a sound amplification slot 1b connecting the outside and the storage cavity 101; the sounding component 7 is arranged in the storage cavity 101 and placed beside the sound amplification slot 1b; the control panel 5 is electrically connected with the sounding component 7.

Specifically, the structures of the sounding component 7 are various, for example, a speaker can be used; A related signal transmission module is installed on the control panel 5. When in use, the audio guide file is uploaded to the control panel 5, and the control panel 5 is used to start and control the speaker to play music or send out other prompt sounds. This design further improves the usability of the multifunctional aroma-diffuser.

It should be noted that the technology of controlling the sounding component 7 to play sound through the control panel 5 is very existing, and will not be described in detail in this application.

Referring to FIG. 3-FIG. 7, the housing 1 is also provided with a charging socket 51 and a plurality of control buttons 52, the charging socket 51 is electrically connected with the control panel 5 to supply power to the control panel 5; the control buttons 52 are electrically connected with the control panel 5 to control the operation status of the aromatherapy component 4, the motor 23, the lamp 3 and the sounding component 7; the user presses the corresponding control button 52 to start and stop the corresponding functional components, which is simple and convenient.

In addition, the manufacturer can also directly arrange a corresponding Bluetooth module on the control panel 5, so as to send instructions to the control panel 5 through the mobile phone, and then control the operating status of the lamp 3, motor 23, fan 43, and sounding component 7.

It should also be noted that although the embodiments of the present application have been shown and described, those skilled in the art can understand that these embodiments can be modified, changed, substituted, amended in various ways without departing from the principle and spirit of the present application.

What is claimed is:

1. A multifunctional aroma-diffuser, comprising:
a housing (1), the housing (1) is provided with a storage cavity (101) and a liquid storage cavity (102) for containing the mixed liquid of essential oil, and the housing (1) is also provided with a light transmission opening (103) and a mist discharge port (104) corresponding to the liquid storage cavity (102);
a lighting auxiliary component (2), the lighting auxiliary component (2) comprises a projection lens (21), a refracting lens (22) and a motor (23); the projection lens (21) is arranged in the light transmission opening (103), the refracting lens (22) is horizontally arranged in the storage cavity (101) and corresponds to the projection lens (21); the motor (23) is fixed in the storage cavity (101) and is drivingly connected to the refracting lens (22), so as to drive the refracting lens (22) to rotate;
a lamp (3), the lamp (3) is arranged in the storage cavity (101), and light emitted by the lamp (3) passes through the refracting lens (22) and the projection lens (21) in sequence;
an aromatherapy component (4), the aromatherapy component (4) is arranged in the housing (1) and configured to atomize the essential oil mixture in the liquid storage cavity (102);
a control panel (5), the control panel (5) is arranged in the storage cavity (101) and is electrically connected with the motor (23), the lamp (3) and the aromatherapy component (4).

2. The multifunctional aroma-diffuser according to claim 1, wherein the projection lens (21) and the refracting lens (22) are both made of transparent plastic or glass material; the refracting lens (22) is provided with a plurality of integrally formed refracting blocks (220); the projection lens (21) is a hemispherical sheet, and a plurality of refracting grooves (210) are uniformly arranged on the projection lens (21).

3. The multifunctional aroma-diffuser according to claim 1, wherein the housing (1) comprises a base (11) and a cover (12), the storage cavity (101) and the liquid storage cavity (102) are arranged on the base (11), and the base (11) is provided with a through port (111) connected to the storage cavity (101) and a liquid inlet (112) connected to the liquid storage cavity (102); the light transmission opening (103) and the mist discharge port (104) is arranged on the cover (12), and the cover (12) is detachably connected to the base (11) to cover the port (111) and the liquid inlet port (112), so that the through port (111) and liquid inlet port (112) respectively correspond to the light transmission opening (103) and the mist discharge port (104);
the motor (23) is fixed in the storage cavity (101) and placed at the through port (111), and the refracting lens (22) is arranged on an output shaft of the motor (23) and corresponds to the through port (111), so that the light from the lamp (3) can be smoothly irradiated onto the projection lens (21) arranged at the light transmission opening (103).

4. The multifunctional aroma-diffuser according to claim 3, wherein the lighting auxiliary component (2) further comprises a fixing frame (24), and the fixing frame (24) is arranged in the storage cavity (101) and placed at the through port (111), the motor (23) is arranged on the fixing frame (24).

5. The multifunctional aroma-diffuser according to claim 3, wherein the lighting auxiliary component (2) further comprises a sealing lens (25), and the sealing lens (25) is arranged at the through port (111) to block the through port (111); the light generated by the lamp (3) sequentially passes through the refracting lens (22) and the sealing lens (25) to the projection lens (21).

6. The multifunctional aroma-diffuser according to claim 1, wherein the lamp (3) is a condenser lens with LED lamps.

7. The multifunctional aroma-diffuser according to claim 3, wherein the aromatherapy component (4) comprises a bracket (41) and an atomizing sheet (42), and an oil hole (102a) is provided on bottom of the liquid storage cavity (102); the bracket (41) is arranged in the storage cavity (101) and placed at the oil hole (102a), the atomizing sheet (42) is arranged on the bracket (41) and blocks the oil hole (102a); the atomizing sheet (42) is also electrically connected with the control panel (5) to atomize the essential oil mixture entering the oil hole (102a), and the fragrance mist formed after the essential oil mixture is atomized is discharged to the outside through the mist discharge port (104).

8. The multifunctional aroma-diffuser according to claim 7, wherein the aromatherapy component (4) further comprises a fan (43), and a cavity cover (6) is arranged on the cover (12), and the cavity cover (6) is provided with an air nozzle (60) connected to the mist discharge port (104);
when the cover (12) is connected to the base (11), the cavity cover (6) blocks the liquid inlet port (112), and the cover (12) and the base (11) enclose to form an air flow cavity (120); the base (11) is provided with an air guide hole (11d) connecting the storage cavity (101) and the air flow cavity (120), and the cavity cover (6) is provided with an air introduction port (6d) connecting the liquid storage cavity (102) and the air flow cavity (120);
the base (11) is also provided with an air inlet slot (1a) connecting the outside and the storage cavity (101); the fan (43) is arranged in the storage cavity (101) and placed beside the air inlet slot (1a), the control panel (5) is electrically connected to the fan (43); the fan (43) is configured to pump the air from the outside into the liquid storage cavity (102), the fragrance mist in the liquid storage cavity (102) can flow from the air nozzle (60) and the mist discharge port (104) to the outside.

9. The multifunctional aroma-diffuser according to claim 1, further comprising a sounding component (7), and the housing (1) is also provided with a sound amplification slot (1b) connecting the outside and the storage cavity (101); the sounding component (7) is arranged in the storage cavity (101) and is placed beside the sound amplification slot (1b); the control panel (5) is electrically connected to the sounding component (7).

10. The multifunctional aroma-diffuser according to claim 9, wherein a charging socket (51) and a plurality of control buttons (52) are also provided on the housing (1), and the charging socket (51) is electrically connected with the control panel (5) to supply power to the control panel (5); the control button (52) is electrically connected to the control panel (5) to control the operation state of the aromatherapy component (4), the motor (23), the lamp (3) and the sounding component (7).
